(19) United States
(12) Patent Application Publication
(10) Pub. No.: US 2004/0015033 A1
(43) Pub. Date: Jan. 22, 2004

(54) Method for producing squalene

(76) Inventors: Rudolf Steiner, Erlangen (DE); Martin Drescher, Fuerth (DE); Mehdi Bonakdar, Liepvre (FR); Wilhelm Johannisbauer, Erkrath (DE)

Correspondence Address: COGNIS CORPORATION, PATENT DEPARTMENT, 300 BROOKSIDE AVENUE, AMBLER, PA, 19002, US

(21) Appl. No.: 10/312,216

(22) PCT Filed: Jun. 21, 2001
(86) PCT No.: PCT/EP01/07208

(30) Foreign Application Priority Data
Jun. 30, 2000 (DE) 10031994.7

(51) Int. Cl. C07C 11/21

(52) U.S. Cl. 585/809

(57) ABSTRACT

A process for making squalene involving: (a) providing a hydrocarbon-containing intermediate product derived from a vegetable oil; (b) providing an extractant; and (c) extracting squalene-containing starting material from the hydrocarbon-containing intermediate product to form a squalene-containing raffinate phase and an extract phase.

### FIELD OF THE INVENTION

This invention relates generally to cosmetic oil components and, more particularly, to a process for the production of squalene by extraction of vegetable oil residues.

### PRIOR ART

Squalane (2,6,10,15,19,23-hexamethyl-2,6,10,14,18,22-tetracosane) belongs to the group of acyclic triterpenes, is usually obtained by hydrogenation of squalene (2,6,10,15,19,23-hexamethyl-2,6,10,14,18,22-tetracoshexaene) and is used as a particularly non-polar oil for cosmetic and pharmaceutical applications. Although the unsaturated parent compound, i.e. squalane, can be synthesized, for example by reaction of hexaphenyl-1,4-butanediyl diphosphonium dibromide with 6,10-dimethyl-5,9-undecadien-2-one, the reaction involves considerable outlay on equipment. Accordingly, squalene or squalane produced in this way is very expensive, especially since the degree of purity required for commercial application is very difficult to achieve.

For this reason, squalene or squalane is normally produced from natural sources such as, for example, shark liver oil or distillates accumulating in the production of vegetable oils, especially olive oil. Unfortunately, squalane from marine animals, which can be obtained for example by extraction, is unsuitable for cosmetic or pharmaceutical application because, despite deodorizing measures, it generally has a troublesome odor and contains unwanted impurities. Particulars of the differences between animal and vegetable squalane (phytosqualane) can be found in A. Gasparoli's Review in Riv. Intaliana, Sostanze Grasse, Vol. LXXIII, pag 293 (1996).

The production of vegetable squalane also belongs in principle to the prior art. Spanish patent application ES 2002428 (Hispano Quìmica) relates to a process for the production of vegetable squalane in which a pressing residue from the production of olive oil is first partly hydrogenated to give a mixture of squalene, squalane, hydrocarbons and unsaturated fatty acids. In a second step, this mixture is converted by fractional crystallization into a liquid concentrate of which the free fatty acids are saponified by addition of a base and removed. The remaining fraction is then hydrogenated, squalene being converted into squalane. Finally, the paraffins still present in the fraction are removed by distillation. Spanish patent application ES 2011259 (Hispano Quìmica) describes a similar process for the production of phytosqualane in which the acidic pressing residue is freed from fatty acids by distillation and saponification, the residue is hydrogenated, the paraffins are frozen out (“winterizing”) and the pure phytosqualane is obtained in a final step by distillation. Finally, Spanish patent ES 2063697 (Hispano Quìmica) proposes carrying out the deparaffination by washing with sulfuric acid. A similar process is the subject of German patent DE 19830171 C1 (Cognis). Japanese patent application JP-A Hei 09/176057 (Koyo Fine Chemicals) relates to a process for the production of squalane in which a fraction accumulating in the purification of olive oil by distillation, which contains 35% by weight of squalene and 50% by weight of free fatty acids, is freed from the acids and hydrogenated. The squalane-containing fraction is then dissolved in isopropyl alcohol and deparaffinized by addition of urea. After removal of the solvent, the squalane is obtained in a purity of 93%. Another process for the production of vegetable squalanes is known from Japanese patent application JP-A Hei 06/306387 (Nisshin Oil Mills). In this process, residues of squalene-containing oils such as, for example, olive oil, soybean oil or palm oil are first hydrogenated and then purified by fractional crystallization. In addition, it is proposed in Japanese patent application JP-A Hei 06/306388 (Nisshin Oil Mills) to take up the acidic distillates in organic solvents after the hydrogenation step and to filter them under pressure through molecular sieves. Finally, German patent application DE 4316620 A1 (Müller Extrakt) describes a multistage process for the production of vegetable squalene in which residues from the production of olive oil are saponified, hydrolyzed, the fatty acids are esterified and the squalene is obtained by extraction with supercritical or subcritical gases. However, this process is equipment-intensive on account of its many individual steps.

Accordingly, the problem addressed by the present invention was to provide a new, simple and environmentally friendly process for the production of the intermediate product squalene which would be distinguished in particular by the fact that the target product is obtained in high purity without any need to use organic solvents.

### DESCRIPTION OF THE INVENTION

The present invention relates to a process for the production of squalene in which hydrocarbon-containing intermediate products from the processing of vegetable oils are extracted with a compressed gas, the squalene accumulating in the raffinate while the impurities are removed as the extract.

It has surprisingly been found that, by applying extraction technology to selected starting materials, squalene can be obtained in purities of more than 80% by weight in a single process step. The process according to the invention provides for non-heat-intensive and hence energy-friendly and environmentally friendly treatment of the educts in which no solvents whatsoever are used. The extractants used are nontoxic and neither inflammable nor corrosive and are inexpensively available in high purity. The squalene is obtained in strictly solvent-free form while the extractant can easily be recovered and then recycled.

Starting Materials

Suitable starting materials for the production of the squalene are, for example, steamer condensates from the refining of vegetable oils such as, for example, palm oil, palm kernel oil, coconut oil or sunflower oil which have a significant hydrocarbon content. Steamer condensates in the context of the invention are understood to be the products obtained after vegetable oils have been deodorized with steam and the vapors precipitated. Steamer condensates from the refining of olive oil are particularly suitable because they almost exclusively contain unsaponifiable fractions which are easy to extract.

Extraction Process

The extraction of the squalene-containing starting materials can be carried out in known manner. Carbon dioxide is a particularly suitable solvent. Extraction may be carried out in a single stage, for example in a stirred autoclave, or in several stages, for example in a countercurrent column, under a pressure of 60 to 30 and more particularly 100 to 140 bar and at a temperature of 20 to 80 and more particularly 40 to 50° C. The multistage procedure affords the advantage of particularly high squalene contents. Removal of the extract may then be carried out under a pressure of 50 to 80 and more particularly 55 to 60 bar and at a temperature of 60 to 120 and more particularly 75 to 85° C. The squalene may then be hydrogenated to squalane in known manner.

### EXAMPLES

### Example 1

250 g of a steamer condensate consisting of 130 g of squalene and 120 g of residual components (corresponding to a squalene content of 52% by weight) were mixed with 700 g of liquid carbon dioxide in an autoclave at 23° C. A pressure of 147 bar was established. 60 mg of a gas-free sample from the lighter extract phase contained 19.7 mg of squalene (corresponding to 32.8% by weight) while 1.9 g of a gas-free sample from the heavier raffinate phase contained 1.02 g (corresponding to 53.7% by weight).

### Example 2

250 g of a steamer condensate consisting of 138.5 g of squalene and 111.5 g of residual components (corresponding to a squalene content of 55.4% by weight) were mixed with 694 g of supercritical carbon dioxide in an autoclave at 40° C. A pressure of 121 bar was established. 47.6 mg of a gas-free sample from the lighter extract phase contained 13.1 mg of squalene (corresponding to 27.5% by weight) while 1.82 g of a gas-free sample from the heavier raffinate phase contained 1.04 g (corresponding to 57.0% by weight).

### Example 3

250 g of a steamer condensate consisting of 212.5 g of squalene and 37.5 g of residual components (corresponding to a squalene content of 85.0% by weight) were mixed with 851 g of supercritical carbon dioxide in an autoclave at 40° C. A pressure of 147 bar was established. 36.5 mg of a gas-free sample from the lighter extract phase contained 22.3 mg of squalene (corresponding to 61.1% by weight) while 1.56 g of a gas-free sample from the heavier raffinate phase contained 1.35 g (corresponding to 86.3% by weight).

### Example 4

250 g of a steamer condensate consisting of 69.1 g of squalene and 180.9 g of residual components (corresponding to a squalene content of 27.7% by weight) were mixed with 48 g of supercritical carbon dioxide in an autoclave at 80° C. A pressure of 50 bar was established. 100% by weight of a sample of the gas phase consisted of carbon dioxide, indicating that the extractant can be completely regenerated under these conditions.

### Example 5

The extraction of a squalene-rich product was carried out at 40° C./120 bar in a high-pressure column with an effective separation height of 7 m. 10 kg/h of the starting product were introduced at the head of the column. The feed contained 55.4% by weight of squalene so that the feed stream consisted of 5.54 kg/h squalene and 4.46 kg/h impurities. A stream of pure carbon dioxide (220 kg/h) was introduced into the column in countercurrent. An extract stream of in all 223 kg/h was obtained at the head of the column, consisting of 97.8% by weight of carbon dioxide (corresponding to 218 kg/h), 0.51% by weight of squalene (corresponding to 1.13 kg/h of squalene) and 1.69% by weight of impurities (corresponding to 3.78 kg/h). The raffinate was removed from the foot of the column. The raffinate stream of, in all, 6.98 kg/h consisted of 27.3% by weight carbon dioxide (corresponding to 1.91 kg/h), 63.0% by weight squalene (corresponding to 4.41 kg/h) and 9.7% by weight impurities (corresponding to 0.68 kg/h). Accordingly, the gas-free raffinate had a squalene content of 86.6% by weight.

1. A process for the production of squalene in which hydrocarbon-containing intermediate products from the processing of vegetable oils are extracted with a compressed gas, the squalene accumulating in the raffinate while the impurities are removed as the extract.

2. A process as claimed in claim 1, characterized in that steamer distillates from the refining of vegetable oils are used.

3. A process as claimed in claim 1 and/or 2, characterized in that steamer distillates from the refining of olive oil are used.

4. A process as claimed in at least one of claims 1 to 3, characterized in that carbon dioxide is used as the extractant.

5. A process as claimed in at least one of claims 1 to 4, characterized in that the extraction is carried out under a pressure of 60 to 300 bar.

6. A process as claimed in at least one of claims 1 to 5, characterized in that the extraction is carried out at a temperature of 20 to 80° C.

7. A process as claimed in at least one of claims 1 to 6, characterized in that removal of the extract is carried out under a pressure of 50 to 80 bar.

8. A process as claimed in at least one of claims 1 to 7, characterized in that removal of the extract is carried out at a temperature of 60 to 120° C.

9. A process as claimed in at least one of claims 1 to 8, characterized in that the extraction is carried out in one or more stages.

10. The use of the squalene obtainable by the process claimed in claims 1 to 9 for the production of squalane.
